(19) United States
(12) Patent Application Publication
(10) Pub. No.: US 2008/0287987 A1
(43) Pub. Date: Nov. 20, 2008

(54) Dispensing system for tissue sealants

(75) Inventors: Edward S. Boyden, Cambridge, MA (US); Roderick A. Hyde, Redmond, WA (US); Muriel Y. Ishikawa, Livermore, CA (US); Eric C. Leuthardt, St Louis, MO (US); Nathan P. Myhrvold, Medina, WA (US); Dennis J. Rivet, St. Louis, MO (US); Michael A. Smith, Phoenix, AZ (US); Clarence T. Tegreene, Bellevue, WA (US); Thomas A. Weaver, San Mateo, CA (US); Charles Whitmer, North Bend, WA (US); Lowell L. Wood, JR., Bellevue, WA (US); Victoria Y.H. Wood, Livermore, CA (US)

Correspondence Address: Searete LLC;Suite 110, 1756 - 114th Ave. S.E., Bellevue, WA, 98004, US

(73) Assignee: Searete LLC, a limited liability corporation of the State of Delaware

(21) Appl. No.: 11/818,884

(22) Filed: Jun. 15, 2007

Related U.S. Application Data
(63) Continuation-in-part of application No. 11/804,219, filed on May 16, 2007.

(51) Int. Cl. A61B 17/03

(52) U.S. Cl. 606/214; 227/175.1

(57) ABSTRACT

The present invention discloses a medical device comprising a dispenser adapted to dispense a tissue sealant or one or more precursor compounds, the dispenser being operably coupled to a surgical stapler, which in turn is configured to deliver one or more surgical staples or fasteners in an area of bodily tissue approximation surface. Furthermore, the medical device comprises a dispenser adapted to dispense a tissue sealant or one or more precursor compounds thereof, the dispenser being operably coupled to a surgical cutter. Additionally or alternatively the medical; device can include a microactuation mechanism, whereby the dispenser dispenses the tissue sealant or one or more precursor compounds at a surgical cutting site or in a proximity thereof.

### CROSS-REFERENCE TO RELATED APPLICATIONS

The present application is related to and claims the benefit of the earliest available effective filing date from the following listed application (the “Related Applications”) (e.g., claims earliest available priority dates for other than provisional patent applications or claims benefits under 35 USC §119(e) for provisional patent applications, for any and all parent, grandparent, great-grandparent, etc. applications of the Related Application).

### RELATED APPLICATIONS

For purposes of the USPTO extra-statutory requirements, the present application constitutes a continuation-in-part of U.S. patent application Ser. No. 11/804,219, entitled STEERABLE SURGICAL STAPLER, naming Edward S. Boyden, Roderick A. Hyde, Muriel Y. Ishikawa, Eric C. Leuthardt, Nathan P. Myhrvold, Dennis J. Rivet II, Michael A. Smith, Clarence T. Tegreene, Thomas A. Weaver, Charles Whitmer, Lowell L. Wood Jr., Victoria Y. H. Wood as inventors, filed May 16, 2007 which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to United States Patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

The present application relates, in general, to devices, methods or systems for dispensing tissue sealants for purposes of medical treatment.

### SUMMARY

An aspect of the invention includes a medical device comprising a dispenser adapted to dispense a tissue sealant or one or more precursor compounds. In an embodiment, the dispenser is operably coupled to a surgical stapler, which in turn is configured to deliver one or more surgical staples or fasteners in an area of bodily tissue approximation surface. In another embodiment, the one or more surgical staples or fasteners may have at least one coating containing the tissue sealant or the one or more precursor compounds. The one or more surgical staples or fasteners may be either pre-coated or post-coated with the tissue sealant or the one or more precursor compounds. Additionally or alternatively, one or more surgical staples or fasteners are either partially coated or fully coated with the tissue sealant or the one or more precursor compounds. In an embodiment the tissue sealant is formed through at least one reaction that includes one or more of the one or more precursor compounds. The at least one reaction includes one or more of the following: a reaction or a reaction with endogenous substrates, a photoreaction with either internal bodily photons or photoreactions with photons external to a bodily tissue or a thermally-driven reaction or a catalytically-activated reaction. In another embodiment, the photo reaction utilizes a photon source operatively coupled to the medical device. Furthermore, the photo reaction utilizes a photon source external to the medical device. In another embodiment, the dispenser is operably configured to dispense microfluidic amounts of the tissue sealant or the one or more precursor compounds. In yet another embodiment, dispenser includes one or more micropipettes that dispense a tissue sealant or one or more precursor compounds. In still another embodiment, the dispenser includes one or more micron-sized outlet ports that dispense a tissue sealant or one or more precursor compounds.

Additionally or alternatively, the dispenser includes one or more microchips containing the tissue sealant or the one or more precursor compounds. In an embodiment, the dispenser includes one or more arrays of microchips containing more than one type of tissue sealant or the one or more precursor compounds. In another embodiment, dispenser includes one or more arrays of micropipettes that dispense more than one type of tissue sealant or the one or more precursor compounds. In yet another embodiment, the dispenser dispenses the tissue sealant or the one or more precursor compounds in a manner that blocks or seals or adheres to holes formed by the one or more surgical staples or fasteners. In still another embodiment, the dispenser dispenses the tissue sealant or the one or more precursor compounds in a manner whereby the one or more surgical staples or fasteners are covered with the tissue sealant or the one or more precursor compounds. Additionally or alternatively, the tissue sealant or the one or more precursor compounds include at least one of the following: antibacterial agents, anti-infection agents, angiogenic factors, growth factors, blood coagulants, antimicrobial agents, pharmaceuticals, drugs or compounds. In an embodiment, the tissue sealant or the one or more precursor compounds reduces scar formation in body tissue. Furthermore, the tissue sealant or the one or more precursor compounds include at least one of an acrylic acid-derivative. In a further embodiment, the tissue sealant or the one or more precursor compounds include at least one of a gel, a cream, a liquid, a fluid, a semi-solid or solid. In one embodiment, the tissue sealant or the one or more precursor compounds include at least one of a hydrogel, an alginate, a zymogen, a glutaraldehyde-treated protein, a cross-linked protein, a cross-linked carbohydrate or a cross-linked fatty acid derivative. In another embodiment, the tissue sealant or the one or more precursor compounds include a volume-expanding substance. In still another embodiment, the staples or fasteners contain at least one coating of the tissue sealant or the one or more precursor compounds on at least one prong of the staples or fasteners. In yet another embodiment, the staples or fasteners contain at least one coating of the tissue sealant or the one or more precursor compounds on at least one crown of the staples or fasteners.

Another embodiment of the medical device further comprises at least one sensor. Furthermore, at least one sensor is configured to regulate the amount of the tissue sealant or the one or more precursor compounds that are dispensed by the dispenser. Additionally or alternatively, the at least one sensor is configured to regulate at least one type of the tissue sealant or the one or more precursor compounds that are dispensed by the dispenser. In an embodiment, the at least one sensor is adapted to sense the amount or level of the tissue sealant or the one or more precursor compounds that are stored in the medical device. In a further embodiment, the at least one sensor includes a proximity detector. The proximity detector may provide a signal or datum pertaining to a position of the one or more surgical staples or fasteners. In an embodiment, the signal or datum may be a homing-type signal. The homing-type signal may be communicated to the dispenser to home-in the dispenser on to staple or fastener-containing sites. In one embodiment, the homing-type signal is communicated to the dispenser to dispense a suitable therapeutic amount, for wound healing purposes, of the tissue sealant or the one or more precursor compounds at a location of the staples or fasteners-containing sites. A further embodiment of the medical device includes a microactuation mechanism that is operably coupled to the dispenser. The microactuation mechanism may be driven by energy generated from an energy module. An embodiment of the energy module includes at least one of the following items: a battery, a capacitor, a fuel cell, a mechanical energy storage device, a solar cell or a fluid energy storage device. In one embodiment, the microactuation mechanism is driven by energy generated from an energy source external to a body. In another embodiment, the microactuation mechanism includes at least one of a pressurized gas canister or cartridge, a spring, a lever, an explosive charge, a piezoelectric actuator, an electric motor, an electroactive polymer, a piezoelectric material or a solenoid. In other embodiments, the microactuation mechanism may be driven by energy reception that includes at least one of an electrical conductor, electromagnetic radiation, fiber optics, fluid flow, material, magnetic induction, acoustic energy, mechanical work or thermal work. Further embodiments provide that the dispenser include at least one micropump. In one embodiment, the micropump is driven by energy derived from at least one of a battery, a capacitor, a fuel cell, a mechanical energy storage device, a solar cell, a piezoelectric material or a fluid energy storage device. Furthermore, the micropump is driven by energy derived from one or more biological metabolites in a body. In some embodiments, the one or more biological metabolites include at least one of the following: a nucleoside, a sugar, a nucleoside phosphate, a nicotinic acid derivative, a nucleotide, a co-enzyme, a vitamin, a peptide, a protein, an amino acid, a carbohydrate, a lipid, a glycolipid, a peptidoglycan, a chromogenic compound, a photo-activatable compound, photoreceptor or a thin-film.

A further aspect of the invention provides a medical device comprising a dispenser adapted to dispense a tissue sealant or one or more precursor compounds thereof, the dispenser being operably coupled to a surgical cutter. Furthermore, the medical device contains an actuation mechanism, whereby the dispenser dispenses the tissue sealant or one or more precursor compounds at a surgical cutting site or in a proximity thereof. In one embodiment, the surgical cutting site has at least one coating containing the tissue sealant or the one or more precursor compounds. In a further embodiment, the surgical cutting site is either partially coated or fully coated with the tissue sealant or the one or more precursor compounds. In another embodiment, the surgical cutter is pre-coated with the tissue sealant or the one or more precursor compounds. In yet another embodiment, the surgical cutter is pre-coated with the tissue sealant or the one or more precursor compounds. In still another embodiment, the tissue sealant is formed through at least one reaction that includes one or more precursor compounds. Additionally or alternatively, at least one reaction includes one of a reaction or a reaction with endogenous substrates, a photo reaction with either an internal bodily photons or photons external to a bodily tissue or a thermally-driven reaction or a catalytically-activated reaction. In one embodiment, the photo reaction utilizes a photon source operatively coupled to the medical device. The photo reaction may utilize a photon source external to the medical device. In another embodiment, the dispenser is operably configured to dispense microfluidic amounts of the tissue sealant or the one or more precursor compounds. In an additional or an alternative embodiment, the dispenser includes one or more micropipettes that dispense a tissue sealant or the one or more precursor compounds. Another embodiment provides for the dispenser to include one or more micron-sized outlet ports that dispense the tissue sealant or the one or more precursor compounds. In yet another embodiment, the dispenser includes one or more microchips containing the tissue sealant or the one or more precursor compounds. The dispenser may further include one or more arrays of microchips containing more than one type of the tissue sealant or the one or more precursor compounds. Further embodiments may include the dispenser containing one or more arrays of micropipettes that dispense more than one type of tissue sealant or the one or more precursor compounds. In some embodiments, the dispenser dispenses the tissue sealant or the one or more precursor compounds in a manner that blocks or seals or adheres to cuts made by the surgical cutter. In another embodiment, the dispenser dispenses the tissue sealant or the one or more precursor compounds in a manner whereby one or more surgical cuts are covered with the tissue sealant or the one or more precursor compounds. In other embodiments, the tissue sealant or the one or more precursor compounds include at least one of antibacterial agents, anti-infection agents, angiogenic factors, growth factors, blood coagulants, antimicrobial agents, pharmaceuticals, drugs or compounds. In some embodiments, the tissue sealant or the one or more precursor compounds include at least one of an acrylic acid-derivative. Furthermore, the tissue sealant or the one or more precursor compounds include at least one of a gel, a cream, a liquid, a fluid, a semi-solid or solid. The tissue sealant or the one or more precursor compounds may include at least one of a hydrogel, an alginate, a zymogen, a glutaraldehyde-treated protein, a cross-linked protein, a cross-linked carbohydrate or a cross-linked fatty acid derivative. Furthermore, the tissue sealant or the one or more precursor compounds may also include a volume-expanding substance. One embodiment provides that the surgical cutter make cuts that contain at least one coating of the tissue sealant or the one or more precursor compounds. In one embodiment, the medical device further comprises of a sensor. The at least one sensor may be configured to regulate the amount of the tissue sealant or the one or more precursor compounds that are dispensed by the dispenser. Furthermore, the at least one sensor is configured to regulate at least one type of the tissue sealant or the one or more precursor compounds that are dispensed by the dispenser. The at least one sensor may be adapted to sense the amount or level of the tissue sealant or the one or more precursor compounds that are stored in the medical device. In one embodiment the at least one sensor includes a proximity detector. The proximity detector may provide a signal or datum pertaining to a position of the dispenser or the surgical cutting site. Additionally or alternately, the proximity detector provides a signal or datum pertaining to a position of the dispenser or the surgical cutting site. Furthermore, the signal or datum may be a homing-type signal. An embodiment provides that the homing-type signal is communicated to the dispenser to home-in the dispenser on to a location or site of a surgical cut. In another embodiment, the homing-type signal is communicated to the dispenser to dispense a suitable therapeutic amount of the tissue sealant or the one or more precursor compounds at the location or site of the surgical cut. According to another embodiment, the microactuation mechanism is driven by energy generated from an energy module. Still another embodiment provides a microactuation mechanism that is driven by energy generated from an energy source external to a body. The energy module may include at least one of a battery, a capacitor, a fuel cell, a mechanical energy storage device, a solar cell or a fluid energy storage device. Additionally or alternatively, the microactuation mechanism includes at least one of a pressurized gas canister or cartridge, a spring, a lever, an explosive charge, a piezoelectric actuator, an electric motor, an electroactive polymer, a piezoelectric material or a solenoid. In some embodiments, the microactuation mechanism is driven by energy reception that includes at least one of an electrical conductor, electromagnetic radiation, fiber optics, fluid flow, material, magnetic induction, acoustic energy, mechanical work or thermal work. In an embodiment the dispenser includes at least one micropump. In a further embodiment, the micropump is driven by energy derived from at least one of a battery, a capacitor, a fuel cell, a mechanical energy storage device, a solar cell, a piezoelectric material or a fluid energy storage device. In another embodiment, the micropump is driven by energy derived from one or more biological metabolites in a body. In yet another embodiment, the one or more biological metabolites include at least one of a nucleoside, a sugar, a nucleoside phosphate, a nicotinic acid derivative, a nucleotide, a co-enzyme, a vitamin, a peptide, a protein, an amino acid, a carbohydrate, a lipid, a glycolipid, a peptidoglycan, a chromogenic compound, a photo-activated compound, a photoreceptor or a thin-film.

Another aspect of the invention provides for a medical device comprising a dispenser adapted to dispense a tissue sealant or one or more precursor compounds, the dispenser being operably coupled to an autosuturing device. Additionally, the medical device comprises a microactuation mechanism, whereby the dispenser dispenses the tissue sealant or one or more precursor compounds at a suturing site or in proximity thereof. In an embodiment, the suturing site has at least one coating containing the tissue sealant or the one or more precursor compounds. Furthermore, the suturing site is either partially coated or fully coated with the tissue sealant or the one or more precursor compounds. In another embodiment, the suturing site is pre-coated with the tissue sealant or the one or more precursor compounds. In yet another embodiment, the tissue sealant is formed through at least one reaction that includes one or more precursor compounds. In still another embodiment the at least one reaction includes one of a reaction or a reaction with endogenous substrates, a photo reaction with either an internal bodily photons or photons external to a bodily tissue or a thermally-driven reaction or a catalytically-activated reaction. Furthermore, the photo reaction may utilize a photon source operatively coupled to the medical device. Alternatively or additionally, the photo reaction utilizes a photon source external to the medical device. In one embodiment, the dispenser is operably configured to dispense microfluidic amounts of the tissue sealant or the one or more precursor compounds. According to another embodiment, the dispenser includes one or more micropipettes that dispense a tissue sealant or the one or more precursor compounds. Yet another embodiment calls for the dispenser to include one or more micron-sized outlet ports that dispense the tissue sealant or the one or more precursor compounds. Still another embodiment provides for the dispenser that includes one or more microchips containing the tissue sealant or the one or more precursor compounds. Some other embodiments optionally provide that the dispenser includes one or more arrays of microchips containing more than one type of the tissue sealant or the one or more precursor compounds. Additionally or alternatively, dispenser includes one or more arrays of micropipettes that dispense more than one type of a tissue sealant or the one or more precursor compounds. A further embodiment provides for the dispenser that dispenses the tissue sealant or the one or more precursor compounds in a manner that blocks or seals or adheres to holes made by the autosuturing device. A different embodiment provides for a dispenser that dispenses the tissue sealant or the one or more precursor compounds in a manner whereby one or more trocar sites are covered with the tissue sealant or the one or more precursor compounds. In one embodiment, the tissue sealant or the one or more precursor compounds include at least one of antibacterial agents, anti-infection agents, anti-infection agents, angiogenic factors, growth factors, blood coagulants, antimicrobial agents, pharmaceuticals, drugs or compounds. Another embodiment provides that the tissue sealant or the one or more precursor compounds include at least one of an acrylic acid-derivative. Yet another embodiment provides that the tissue sealant or the one or more precursor compounds include at least one of a gel, a cream, a liquid, a fluid, a semi-solid or solid. Still another embodiment provides that the tissue sealant or the one or more precursor compounds include at least one of a hydrogel, an alginate, a zymogen, a glutaraldehyde-treated protein, a cross-linked protein, a cross-linked carbohydrate or a cross-linked fatty acid derivative. Furthermore, the tissue sealant or the one or more precursor compounds include a volume-expanding substance. Furthermore, the suturing site contains at least one coating or covering of the tissue sealant or the one or more precursor compounds. In an embodiment, the medical device further comprises at least one sensor. The at least one sensor is configured to regulate the amount of the tissue sealant or the one or more precursor compounds that are dispensed by the dispenser. The at least one sensor may be configured to regulate at least one type of the tissue sealant or the one or more precursor compounds that are dispensed by the dispenser. Alternatively or additionally, at least one sensor is adapted to sense an amount or level of the tissue sealant or the one or more precursor compounds that are stored in the medical device. A further embodiment provides that the at least one sensor includes a proximity detector. In an additional embodiment, the proximity detector provides a signal or datum pertaining to a position of the autosuturing device. In an embodiment, the signal or datum may be a homing-type signal. Additionally or alternatively, the homing-type signal is communicated to the dispenser to home-in the dispenser on to a location or site of a surgical suture. Furthermore, homing-type signal is communicated to the dispenser to dispense a suitable therapeutic amount of the tissue sealant or the one or more precursor compounds at the location or site of a surgical suture. In an embodiment, the microactuation mechanism is operably coupled to the dispenser. In another embodiment, the microactuation mechanism is driven by energy generated from an energy module. The energy module may include at least one of a battery, a capacitor, a fuel cell, a mechanical energy storage device, a solar cell or a fluid energy storage device. Additionally or alternatively, microactuation mechanism includes at least one of a pressurized gas canister or cartridge, a spring, a lever, an explosive charge, a piezoelectric actuator, an electric motor, an electroactive polymer, a piezoelectric material or a solenoid. In one embodiment, the microactuation mechanism is driven by energy generated from an energy source external to a body. In another embodiment, the microactuation mechanism is driven by energy reception that includes at least one of an electrical conductor, electromagnetic radiation, fiber optics, fluid flow, material, magnetic induction, acoustic energy, mechanical work or thermal work. In some embodiments, the dispenser includes at least one micropump. The micropump may be driven by energy derived from at least one of a battery, a capacitor, a fuel cell, a mechanical energy storage device, a solar cell, a piezoelectric material or a fluid energy storage device. Furthermore, the micropump may be driven by energy derived from one or more biological metabolites in a body. Additionally or alternatively, the one or more biological metabolites include at least one of a nucleoside, a sugar, nucleoside phosphate, a nicotinic acid derivative, a nucleotide, a co-enzyme, a vitamin, a peptide, a protein, an amino acid, a carbohydrate, a lipid, a glycolipid, a peptidoglycan, a chromogenic compound, a photo-activated compound, photoreceptor or a thin-film.

Another aspect of the invention involves a method of applying tissue sealant to an area of a tissue approximation surface, which comprises the step of dispensing a selected amount of the tissue sealant or one or more precursor compounds, the dispensing occurring through a dispenser that is operably coupled to a surgical device. In an embodiment of the method, the surgical device is a surgical stapler, a surgical fastener, a trocar device, a surgical cutter or a suturer. Furthermore, the dispensing includes application of the tissue sealant or the one or more precursor compounds in response to at least a single user-initiated actuation of the surgical device. The dispensing includes location of a site of at least one surgical cut, a surgical suture or a trocar site in a body tissue. A further embodiment may include the dispensing step involving moving a dispenser to a location of a site of at least one surgical staple or fastener in a body tissue. Additionally or alternatively, the dispensing includes application of therapeutic amounts of the tissue sealant or the one or more precursor compounds for purposes of inducing anti-infection response, angiogenesis, promoting tissue growth, enhancing blood coagulation, antimicrobial activity, antiviral response or for reducing scar formation and reducing tissue adhesions. The dispensing step may further include application of amounts of the tissue sealant or the one or more precursor compounds that will hold the tissue approximation surface together for wound healing purposes. In an embodiment the dispensing includes pre-coating a staple or fastener with a therapeutic amount of the tissue sealant or the one or more precursor compounds sufficient to promote anti-infection, angiogenesis, tissue growth, blood coagulation, antimicrobiosis or antiviremia. Additionally or alternatively, the dispensing includes coating a staple or fastener following tissue stapling or fastening with a therapeutic amount of the tissue sealant or the one or more precursor compounds sufficient to promote anti-infection, angiogenesis, tissue growth, blood coagulation, antimicrobiosis or antiviremia. In another embodiment, the dispensing includes application of the tissue sealant or the one or more precursor compounds to a prong of a staple or fastener or in a vicinity thereof. In yet another embodiment, the dispensing includes application of the tissue sealant or the one or more precursor compounds on a crown of a staple or fastener. In still another embodiment, the dispensing includes application of the tissue sealant or the one or more precursor compounds in a vicinity of a crown of a staple or fastener. In still another embodiment, dispensing includes application of the tissue sealant or the one or more precursor compounds for purposes of repairing, sealing or welding of blood vessels. In some embodiments the area of a tissue approximation surface includes, by way of example, one of a blood vessel, a nerve, a cartilage, a bone, a stomach, a pulmonary artery, a vein, a thoraco-abdominal cavity, an intestine, a duodenum, a skin, a fascia, a dermis, a muscle, a meningeal layer, a bowel wall or a mucosal layer. Furthermore, the area of a tissue approximation surface may include a first bodily tissue configured to adhere to second bodily tissue. Another embodiment includes, the first and second bodily tissues being secured by a surgical staple or surgical fastener.

A further aspect of the invention involves a medical device comprising a means for dispensing a selected amount of the tissue sealant or the one or more precursor compounds in an area of a tissue approximation surface or at a site therein, the dispensing occurring through a dispenser that is operably coupled to a surgical device. In an embodiment, the medical device is a surgical stapler, a surgical fastener, a trocar device, a surgical cutter or a suturer.

Yet another aspect of the invention includes a grasper serving as an anchor or connector, the grasper configured to elute a drug or a pharmaceutical compound while holding one or more bodily tissues or a portion of a bodily tissue. In an embodiment the grasper includes at least one of a staple, a fastener, a pin, a suture, a cord, a fixture, a filament, a closure device, a clip, a stent, a tie or any deployable tissue grasping construct. Alternatively or additionally, the grasper includes an elutable drug or a pain medication compound or a chemotherapeutic or an antibiotic agent at a site or a location of the holding.

An aspect of the invention includes a medical device comprising a surgical tool, and a dispenser configured to dispense a tissue sealant in a surgical incision, wherein the dispenser is operably coupled to the surgical tool. Additionally or alternatively, the surgical tool can be a surgical stapler or an autosuturing device or a cutter. Furthermore, the dispenser includes a reservoir configured to retain a quantity of the tissue sealant.

Another aspect of the invention includes a method of sealing approximated body tissue comprising dispensing a tissue-sealing amount of a tissue sealant to the approximated tissue, the dispensing occurring via a medical device that comprises a surgical tool operably coupled to a dispenser mechanism, wherein the dispenser mechanism is configured to controllably dispense the tissue sealant.

Still another aspect of the invention includes a method of maintaining and sealing the approximation of tissue surfaces with a medical device, comprising: securing the approximation of the tissue surfaces relative to one another with at least one surgical staple; and dispensing a tissue-sealing amount of a tissue sealant to the secured approximated tissue; wherein the medical device includes a surgical stapler operably coupled to a tissue-sealant dispenser mechanism.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a system-level illustration of an example of a medical device in which, embodiments such as a dispenser, grasping jaws and an example of a force generator mechanism may be implemented;

FIG. 2 is a schematic of tissue sealant-coated surgical staples and fasteners or pins;

FIG. 3 is a schematic of an illustrative embodiment of a dispenser and examples of a tissue sealant, microchips and a micropump;

FIG. 4 is a schematic of an illustrative embodiment of a dispenser. In the drawing, an example of a tissue sealant is illustratively shown to be delivered to sites of prongs in an illustrative surgical staple that is located in an example of a body tissue approximation surface;

FIG. 5 is a schematic of an illustrative embodiment of a dispenser. In the drawing, an example of a tissue sealant is illustratively shown to be delivered to an illustrative crown of a surgical staple that is located in an example of a body tissue approximation surface;

FIG. 6 is a schematic of an illustrative embodiment of a dispenser. In the drawing, an example of a tissue sealant is illustratively shown to be delivered to an illustrative surgical fastener or pin that is located in an example of a body tissue approximation surface;

FIG. 7 is a schematic of an illustrative embodiment of a dispenser with an example of sensors;

FIG. 8 is a schematic of an illustrative embodiment of a dispenser with an example of a sensor and an example of a proximity detector;

FIG. 9 is a system-level illustration of an example of a medical device in which embodiments such as a dispenser, surgical cutter and an example of a force generator mechanism may be implemented;

FIG. 10 illustrates embodiments of an example of an operational flow for dispensing tissue sealant;

FIG. 11 illustrates embodiments of an example of an operational flow for dispensing tissue sealant;

FIG. 12 illustrates embodiments of an example of an operational flow for dispensing tissue sealant;

FIG. 13 illustrates embodiments of an example of an operational flow for dispensing tissue sealant;

FIG. 14 illustrates embodiments of an example of an operational flow for an illustrative area of tissue approximation; and

FIG. 15 illustrates an operational flow for implementing embodiments of an example of a medical device.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

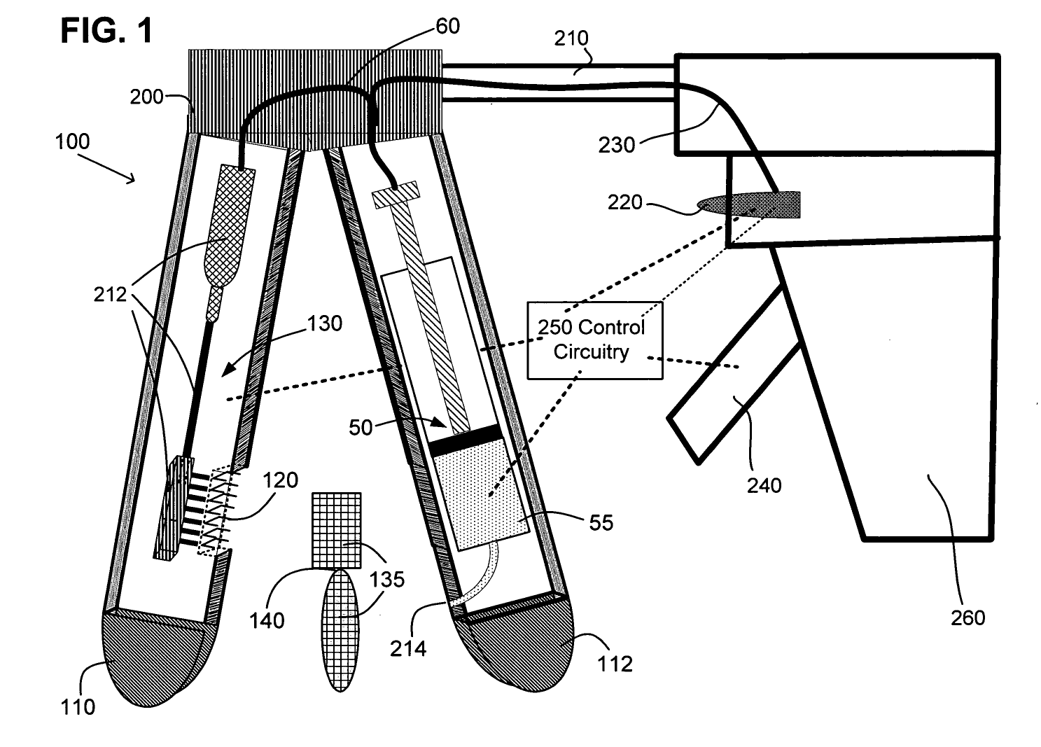

The following disclosure is drawn to a medical device. FIG. 1 is a system-level illustration of a medical device 100. The medical device comprises a dispenser 50 adapted to dispense a tissue sealant 55 or one or more precursor compounds. The dispenser is operably coupled 60 to a surgical stapler 130, which in turn is configured to deliver one or more surgical staples 120 (or fasteners) in an area of bodily tissue 135 approximation surface 140. In some embodiments, the medical device, inter alia, may include at least two grasping jaws 110, 112, which may be connected to each other by hinge 200. In one or more embodiments, the hinge 200 may be connected to a shaft 210, which may be flexually deformable to permit steering of the medical device 100 and to increase maneuverability around anatomical corners or difficult-to-reach anatomical body parts that are normally inaccessible on a straight trajectory. Examples of material that may be employed to increase steerability of the shaft include shape-transforming materials such as shape memory alloy. The shape memory alloy may include for example, titanium, nickel, zinc, copper, aluminum, cadmium, platinum, iron, manganese, cobalt, gallium or tungsten. Alternatively, the shape memory alloy includes Nitinol™ or an electro-active polymer. Alternative embodiments call for at least one shape-transforming material to include at least one mechanically reconfigurable material. Returning to FIG. 1, the shaft 210 is typically connected to a handle member 260 that a user would hold to deploy to use the medical device 100. The user may actuate the stapler 130 or the dispenser 50 by pressing the trigger member 240. The trigger may include a variety of devices such as a push-button mechanism or a mechanical lever or a spring, etc. Those skilled in the art will recognize that the trigger member may actuate the stapler or the dispenser may be operated either simultaneously or synchronously or separately or in combination thereof. In an embodiment, the at least one of the grasping jaws 110 may house various parts of a surgical staple-delivery mechanism 212 while the other grasping jaw 112 may house the dispenser 50. The dispenser may dispense its contents through an outlet port 214 that is configured to dispense selected or graduated amounts of the tissue sealants at suitable sites on or in a bodily tissue 135. In an embodiment, the grasping jaws may operate in a coordinated fashion in the sense that in a typical grasp-and-release cycle stapling operation may be operably coupled to dispensing of a dose of a tissue sealant. Additional embodiments of the medical device 100, may provide that the grasping-and-dispensing operations may be controlled by a regulator 220 that is operably coupled to the grasping jaws via an example of a hard wire 230 device. Alternatively or additionally, the regulator 220 may govern the operations of the dispenser 50 and the stapler 130 via a wireless device (not shown). The medical device may further comprise control circuitry 250 (shown in FIG. 1 illustratively as a box) that may control or regulate various operational components in the medical device. One skilled in the art will appreciate that control circuitry may include hardwired or wireless devices, electrical or electronic components, switching devices, transmitters, receivers, etc.

As used herein, the terms “grasping jaws” or “jaws” include, but are not limited to, any of the various parts or whole of a surgical stapler or parts thereof or similar surgical stapling or anastomosis devices. Illustrative examples of such staplers, stapling devices or anastomosis devices may be those suitable for use in any medical or surgical care including performing end-to-end anastomosis, side-to-side anastomosis, individual ligation, endoscopic or laparoscopic gastro-intestinal operations. Such operations may involve, for example, at least one of a bronchus, a pulmonary artery, a pulmonary vein, a large or small intestine, a stomach, a blood vessel, skin, a fascia, a dermis, a muscle, a meningeal layer, a bowel wall or a mucosal layer. Those skilled in the art will realize that the grasping jaws may be configured such that the shape and size of the grasping surface is altered based on the size and shape of the bodily organs or tissues. In other words, grasping jaws may be constructed in different sizes and shapes to fit the various bodily organs and tissues of patients. Furthermore, one or more grasping jaws may be configured to enter the lumen of tubular organs during anastomosis procedures.

In an embodiment, the terms “stapler” or “fastener” or “cutter” refer to any tool or device or system that is functionally capable of physically bringing together two or more bodily tissues to form at least one tissue approximation surface. The term “tissue approximation surface” includes, inter alia, surfaces created by bringing together tissues or an area of tissue incision or destruction or cutting. Examples of tissue approximation surfaces have been discussed in a filed U.S. patent application Ser. No. 11/788,767 entitled “Systems and Methods for Approximating Surfaces”, which is incorporated herein by reference.

As used herein, the term “grasper” refers to, but is not limited to, at least one of a staple, a fastener, a pin, a suture, a cord, a fixture, a filament, a closure device, a clip, a stent, a tie or any deployable tissue grasping construct.

The terms “bodily”, “body” or “patient”, as used herein, refer to a human or any animal including domestic, marine, research, zoo, farm animals, fowl and sports animals, or pet animals, such as dogs, cats, cattle, horses, sheep, pigs, goats, rabbits, chickens, birds, fish, amphibian and reptile.

The terms “tissue(s)” or “organs”, as used herein, includes any part of a human or animal body. Examples may include, but are not limited to, organs associated with the alimentary canal or digestive tract, pulmonary tract, blood vessels, lumen-containing organs, bones, brain, spine, heart, skin etc.

As used herein, the terms “tissue sealant” or “precursor compounds” include, but are not limited to, glue, adhesive, sealant, fastener, tape, sticky material, biological adhesive material, rope, string or any of the various materials that may be used to hold two or more surfaces together for any length of time. The “tissue sealant” or “precursor compounds” may be in any shape or form including, but not limited to, at least one of a gel, a cream, a liquid, fluid, semi-solid, solid or gaseous state. In one embodiment, the tissue sealant or the one or more precursor compounds include at least one of a hydrogel, an alginate, a zymogen, a glutaraldehyde-treated protein, a cross-linked protein, a cross-linked carbohydrate or a cross-linked fatty acid derivative. Furthermore, tissue sealants can include at least one of the following: antibacterial agents, anti-infection agents, angiogenic factors, growth factors, blood coagulants, antimicrobial agents, pharmaceuticals, drugs or acrylic compounds. In some embodiments, the tissue sealants could be deposited as a coating on any part of a staple, a pin, a fastener or a tie, a cord, a rope, a string, a lasso or on any tissue approximation connector. Deposition of the tissue sealant can be done either during the manufacture, referred to as pre-coating, or after manufacture, referred to as post-coating, of the any of the above devices. Post-coating, inter alia, can occur after the tissue approximation device such as a staple or fastener has been deployed or during the deployment tissue approximation device. Numerous tissue sealants for welding blood vessels have been disclosed by others. For example, cyanoacrylates and alkylacrylates are well-known in the art. United States Patent pre-grant application Nos. 20060147479, 20050228443 and U.S. Pat. Nos. 5,081,282, 6,518,308 and 5,081,282, which are incorporated herein by reference, disclose compositions for tissue sealants. Compounds used for reducing scar formation have been reported, for example in U.S. Pat. Nos. 6,319,942, 6,756,518 and in WIPO pub. No. WO/2000/051566, which are incorporated herein by reference. Those skilled in the art will recognize that different reaction components may be mixed either within the body chamber or may be delivered to the outside to effectuate reactions outside the dispenser or medical device to generate sealants comprising various compositions.

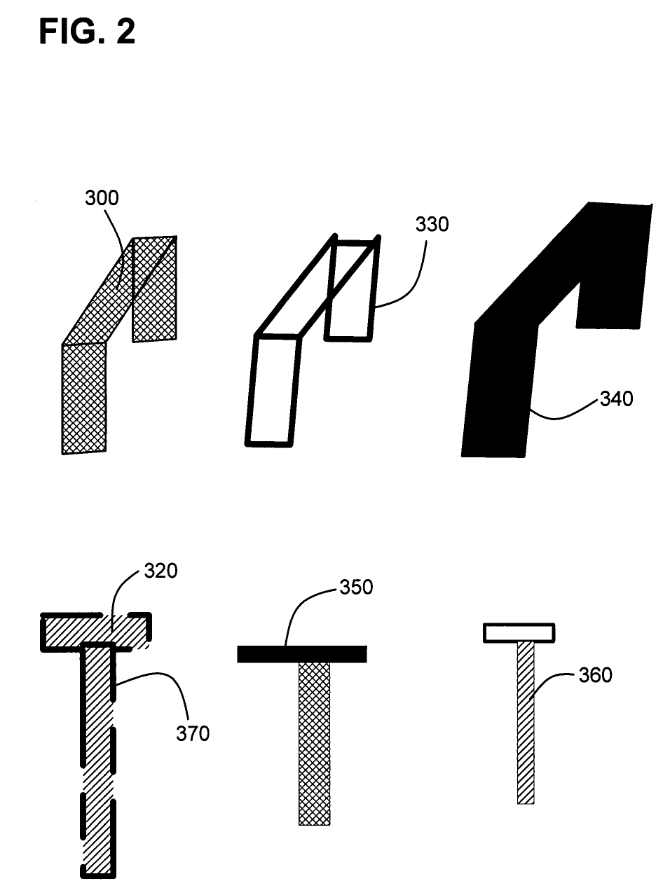

FIG. 2 schematically illustrates some embodiments of surgical staples and fasteners or pins coated with a tissue sealant. Typically, a staple may be partially coated 300 with a tissue sealant or one or more precursor compounds or coated on the edges 330 or fully coated 340. Those skilled in the art will realize that surgical staples may be coated with the sealant on at least a portion of a prong(s) or a crown of a typical staple. Likewise, a pin or a fastener 320 may be partially coated 370 or fully coated on a head portion 350 or a stem portion (prong) 360. One skilled in the art will be aware that fasteners or pins, like staples, are commercially available in different shapes and sizes to suit various surgical needs. Alternatively, pins or fasteners may be custom manufactured with specific materials for specific surgical needs or to fit specific types of medical devices. In some embodiments, the pins, fasteners or staples may be coated with sealants before they are deployed (referred to herein as pre-coating) or coated with sealant following deployment on a tissue surface (post-coating). In a post-coating, the coating may be applied at any time after the manufacture or during the deployment of staple or pin or fastener.

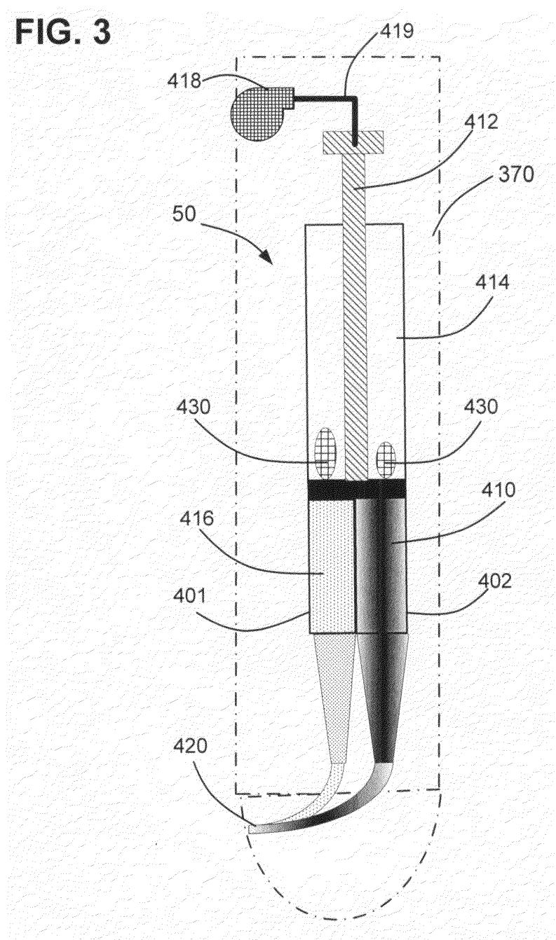

In an embodiment, as illustratively exemplified in FIG. 3, a dispenser 50, may comprise of a body chamber 414 that may be cylindrically shaped or non-cylindrically shaped. The body chamber may be made from any material, typically vinyl or metal. The body chamber may be adapted to hold a piston or plunger 412, which may be used to control the flow of a tissue sealant or precursor compounds 410, 416. In an embodiment, the plunger or piston may be adjusted to regulate a flow rate of the sealant or precursor compounds. Alternatively or additionally in some embodiments, the flow rates may be controlled by a micropump 418 that is connected 419 to the plunger or piston. In one embodiment, the micropump or the plunger may operably control the droplet-velocity of the emerging sealant through a system of microchips 430 implanted within or in the vicinity of the dispenser. One skilled in the art will appreciate that these types of flow-control mechanisms have been described in detail elsewhere and may be readily adapted without undue experimentation to the medical device disclosed herein. For example, U.S. Pat. Nos. 6,720,710 and 7,195,465, and Pre-grant Pub. Nos. 2006/0105453 and 2006/0105453, which are incorporated herein by reference, disclose micropumps and related devices. In alternate or additional embodiments, the body chamber may comprise of multiple compartments 401, 402 that may hold different types of sealants or precursor compounds that may be of various compositions. Those skilled in the art will recognize that different reaction components may be mixed either within the body chamber or may be delivered to the outside to effectuate reactions outside the dispenser or medical device to generate sealants comprising various compositions. For instance in, as shown in an embodiment in FIG. 3, a fused outlet port 420 may be configured to deliver a mixture of precursor compounds to initiate a reaction for developing a sealant to be deposited outside the dispenser 50. One skilled in the art will realize that size of the outlet ports may be adapted to control flow rate of sealants. For example, micron-sized outlet ports 420 may be used to deliver microfluidic amounts of tissue sealants or precursor compounds. In an embodiment, the dispenser may be enclosed in at least one grasping jaw 370 (illustratively shown in outline form in FIG. 3).

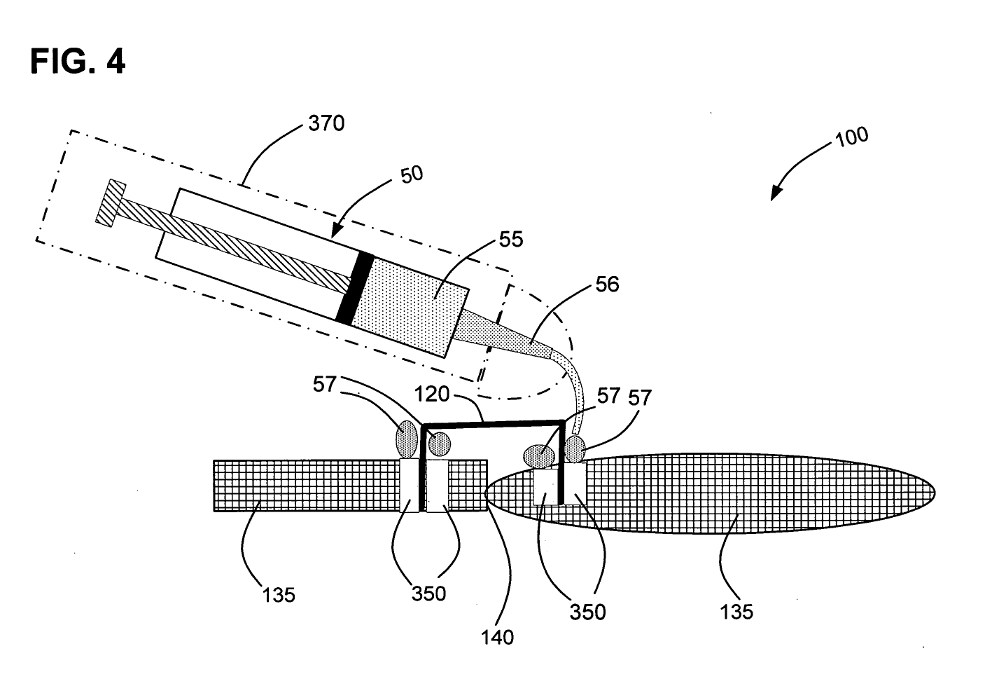

FIG. 4 illustrates an embodiment of a medical device 100 wherein a dispenser 50 includes a micropipette 56 that is configured to dispense a tissue sealant 55 in a manner that blocks or seals or adheres to holes 350 that are formed by a surgical staple 120. In one embodiment the surgical staple is deployed in a surface approximation area 140. The surface approximation area includes, inter alia, an area that is typically formed by the joining of one or more bodily tissues 135. In alternative or additional embodiments, microdroplets 57 of tissue sealant may be delivered at pin-point locations to seal the holes 350 created by the staple 120. One skilled in the art will appreciate that the sealant may be designed and delivered in such a fashion that it may include agents or materials that promote wound healing.

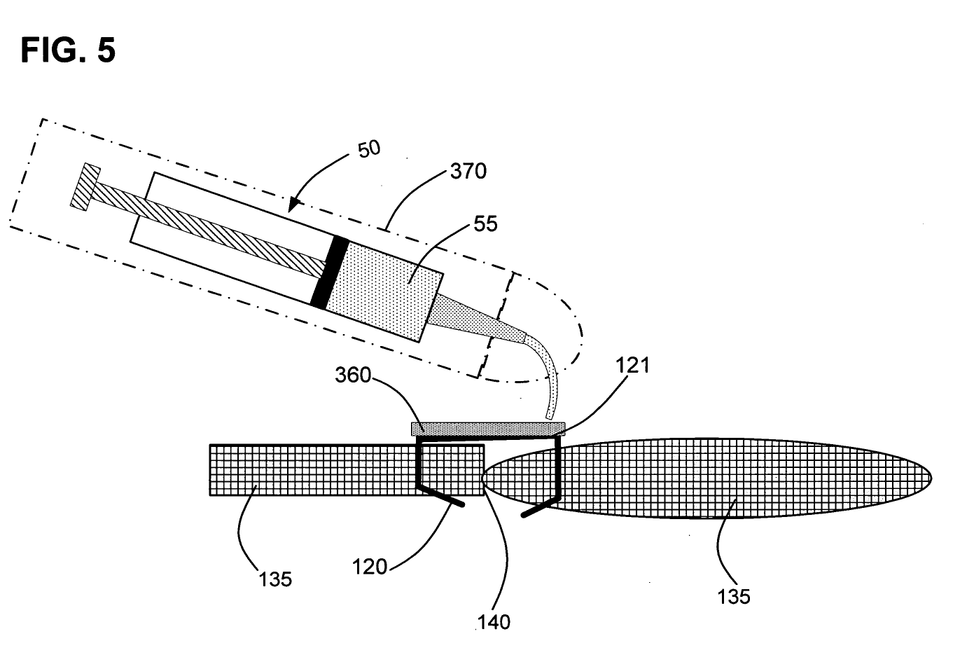

Alternatively or additionally, as shown in FIG. 5, a dispenser 50 may be used to deliver a tissue sealant 55 as coating 360 over the crown 121 of a surgical staple 120. Those skilled in the art will appreciate that coating the staple crown is probably advantageous in surgical operations where a large area of wound healing is required. Examples of this type of operations where rows of staples are used, include, but are not limited to, surgical operations that involve a stomach, a pulmonary artery, mesentery of the abdomen, a thoraco-abdominal cavity, a viscus, an artery, a vein, or any vascular conduit, or any layers of a thoraco-abdominal cavity or a skin. One skilled in the art will also appreciate that coating of staples with adhesive or sealant may be achieved prior to staple delivery (pre-coating) or after stapling operations (post coating). U.S. Pat. Nos. 4,941,623, 4,655,222, 5,027,834, 5,578,031, 5,814,022, 6,126,658, 6,860,895, 7,179,258, and United States Patent application pre-grant publication Nos. 2002/0055701, 20060271041 2004/0111115, which are incorporated herein by reference, provide examples of devices and methods for welding or sealing or cauterizing bodily organs and tissues.

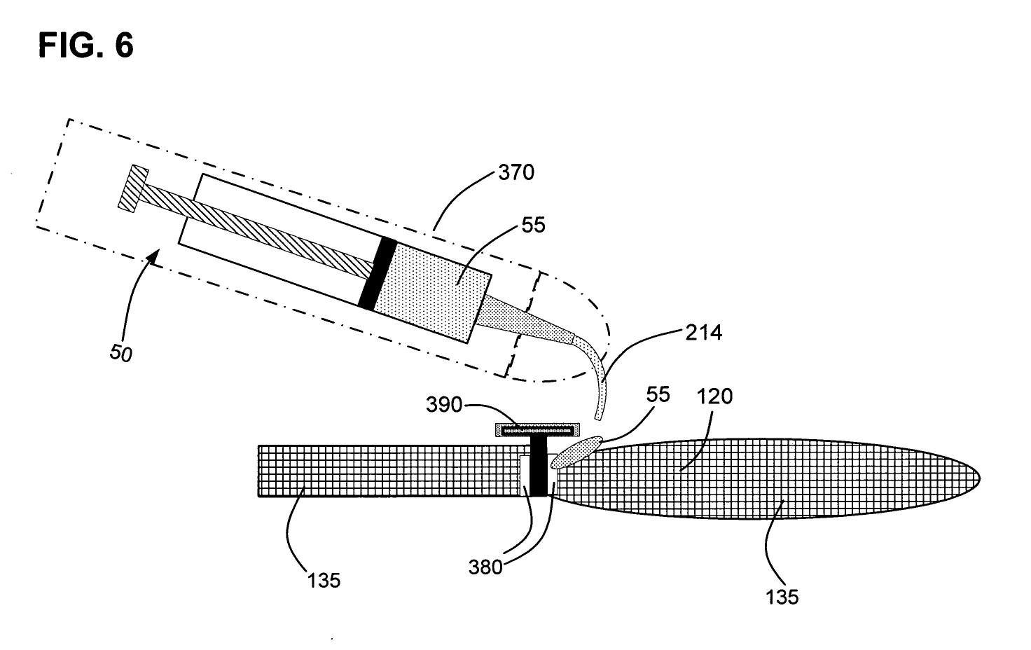

Looking at FIG. 6, there is exemplified a dispensing operation on an illustrative surgical fastener. In this embodiment, a dispenser 50 housed in a grasping jaw (outline) 370 is employed to dispense a tissue sealant 55 at a location of a surgical fastener or pin 390 that has been deployed in an area of a joint 380. In an embodiment, an outlet port 214 may be configured to be protractable or retractable to permit accurate deposition of the sealant. One skilled in the art will appreciate that this joint may include a region wherein two or more bodily tissues 135 have been held together by a surgical fastener or pin 390. Those skilled in the art will further recognize that fasteners or pins (or screws) may be used to join or seal bones or bone fragments. Healing of bone fractures may be promoted through application of wound healing sealants at sites where fasteners have been deployed or at sites of instrumentation and arthrodesis or at sites where other mechanical and structural adjuvants have been applied for body healing. In some embodiments, the sealants may be doped with other agents with properties including, but not limited to, antisepsis, antibacterial or anti-infectious agents and synthetic pharmacological promoters of bone healing or growth e.g. entire family of bone morphogenic proteins. U.S. Pat. Nos. 4,767,044 and 6,830,573, for example, which are incorporated by reference herein, describe fastener devices and applicators.

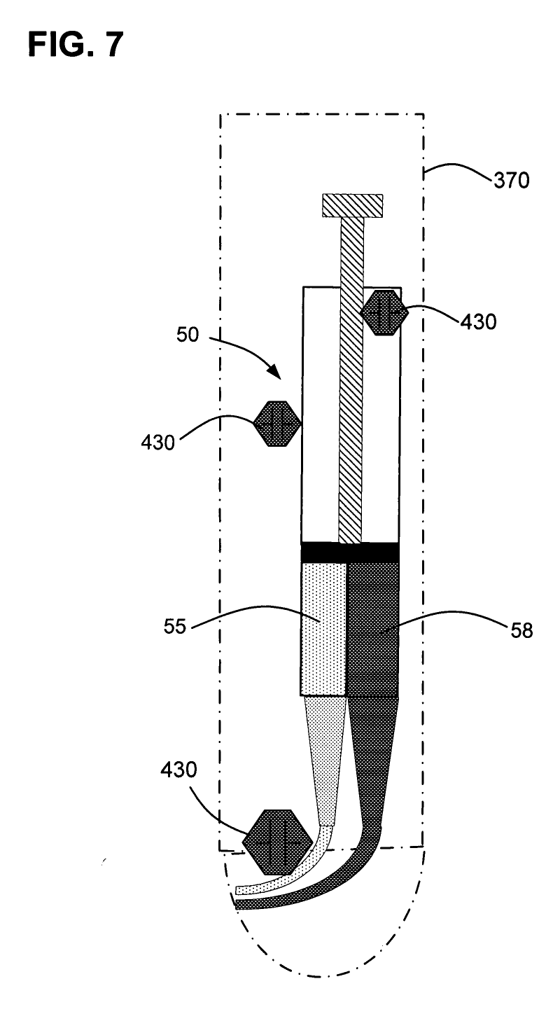

According to FIG. 7, a dispenser 50 may have multiple sensors 430, which may sense, inter alia, the rate of delivery of one type of tissue sealant 55 or another type of sealant 58 (or both) carried by the dispenser. Alternatively or additionally, the sensors may monitor or sense the level and amount of sealant available for dispensing in a dispenser. In an embodiment, the dispenser 50, may be housed in a grasping jaw 370 (as shown in an outline). One skilled in the art will appreciate that during surgery it may be useful for users of the device described herein to possess information regarding the amount of sealant remaining in a dispenser in order to appropriately refill during surgical procedures.

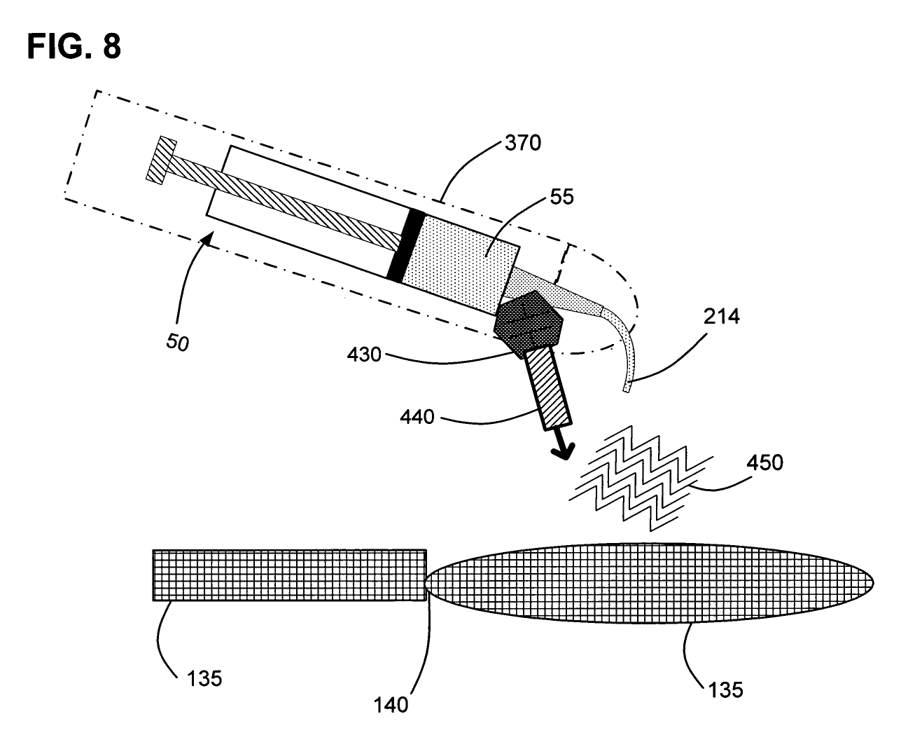

An embodiment of a dispenser 50 having a sensor 430 that is configured to carry a proximity detector 440 is illustrated in FIG. 8. In another embodiment, the proximity detector 440 provides a signal 450 (or datum) to a user pertaining to a position of one or more bodily tissues 135 and tissue approximation surface 140. In alternative embodiments, the proximity detector may be adapted to provide homing type of signal so as to facilitate the positioning of an outlet port 214 in a vicinity of the tissue approximation surface 140 for easy and accurate dispensation of the tissue sealant 55. In additional or alternative embodiments, sensors may provide a valve-like function, controllably regulating the outflow of tissue sealant from the dispenser. In additional or alternative embodiments, the proximity detector may used provide to the user locations of fiducials.

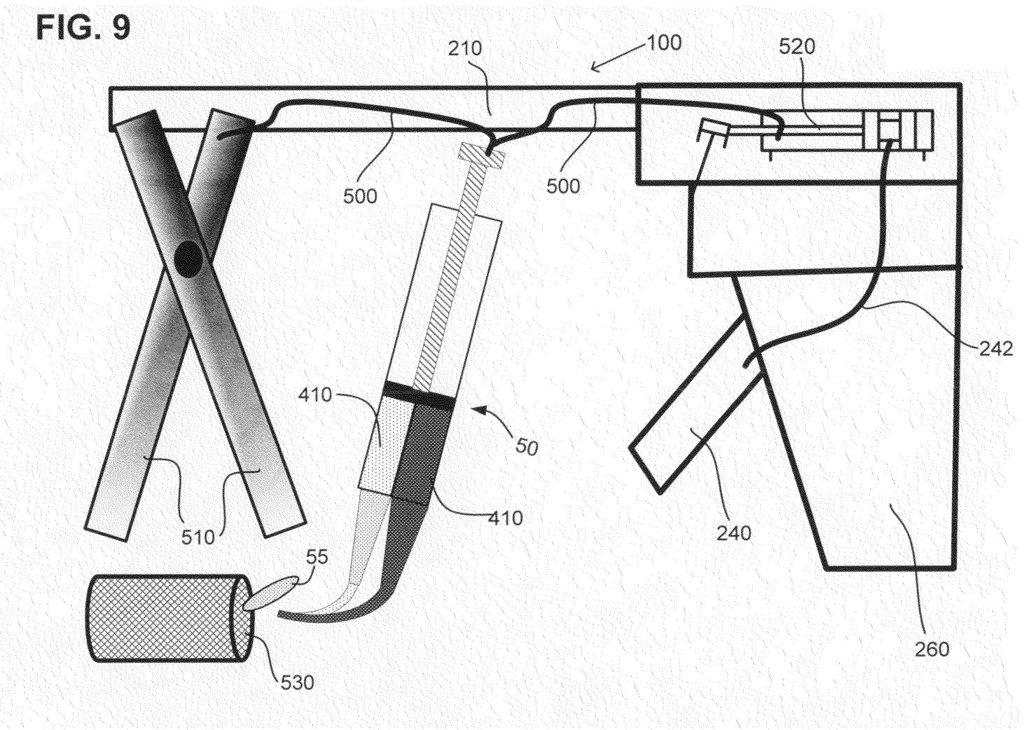

A further aspect of the invention is illustrated in FIG. 9. At a system level, a medical device 100 is shown to have a dispenser 50 that is operably coupled to a surgical cutter 510. In an embodiment, the dispenser may dispense a tissue sealant 55 or one or more precursor compounds 410. One skilled in the art will realize that the operable coupling permits a user to dispense the sealant either simultaneously at the time the surgical cut is made or shortly thereafter. The advantage of this may be that wound healing or promotion of tissue approximation may be achieved with greater efficacy. Additionally or alternately, the dispenser 50 dispenses the tissue sealant 55 or one or more precursor compounds 410 at a surgical cutting site 530, or in proximity thereof. At a system level, the operability of the surgical cutter 510 and the dispenser 50 may be facilitated by connecting the two entities through a hardwire device 500, which in turn may be connected to a microactuation mechanism 520 that controls the operation of the cutter and the dispenser. In an embodiment, the hardwire device 500 may be disposed in a shaft 210 that connects the cutter 510 and a handle member 260. In a further embodiment, a trigger member 240 may be used by a user to activate the microactuation mechanism 520 through a hardwire 242 connection. One skilled in the art will realize that the hardwire connections described above may be replaced without undue experimentation by wireless devices that are commercially available. Typical systems utilizing wireless operations include, inter alia, radio transmitters and receivers, remote controls, computer networks, network terminals, etc., which use some form of energy (e.g., radio frequency (RF), infrared light, laser light, visible light, acoustic energy, etc.) Wireless systems of communication may or may not be “cordless or mobile” and do not preclude hardwiring of systems, and digital or analog systems.

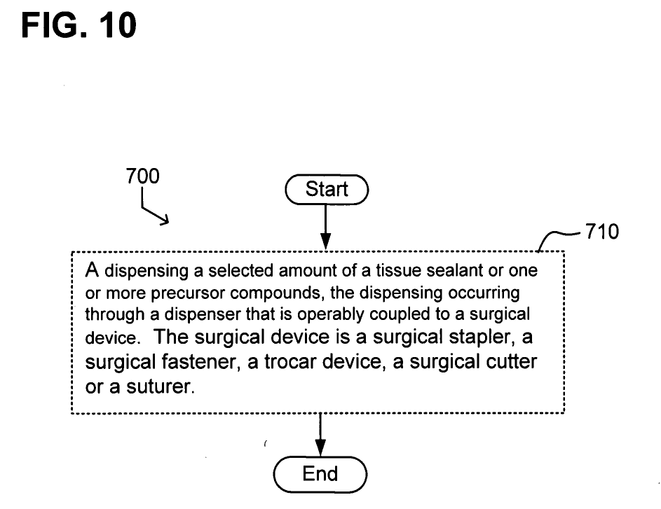

In FIG. 10, according to the present invention there is provided an example of an operational flow 700 for a method of applying a tissue sealant to a body tissue comprising: 710 dispensing a selected amount of a tissue sealant or one or more precursor compounds in an area of a tissue approximation surface or at a site therein, the dispensing step occurring through a dispenser that is operably coupled to a surgical device. Additionally or alternatively, the surgical device is a surgical stapler, a surgical fastener, a trocar device, a surgical cutter or a suturer.

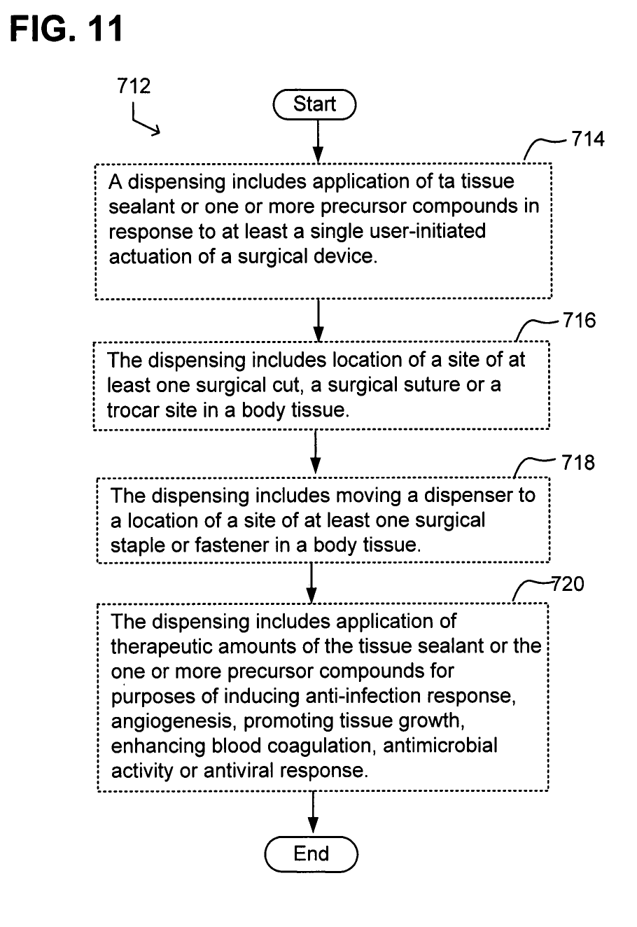

FIG. 11 schematically illustrates embodiments of an example of an operational flow 712 for a dispensing step 714 includes application of a tissue sealant or one or more precursor compounds in response to at least a single user-initiated actuation of a surgical device. Another embodiment of the dispensing step may optionally include 716 location of a site of at least one surgical cut, a surgical suture or a trocar site in a body tissue. Furthermore, the dispensing step 718 includes moving a dispenser to a location of a site of at least one surgical staple or fastener in a body tissue. In another embodiment, the dispensing step 720 includes an application of therapeutic amounts of the tissue sealant or the one or more precursor compounds for purposes of inducing anti-infection response, angiogenesis, promoting tissue growth, enhancing blood coagulation, antimicrobial activity or antiviral response.

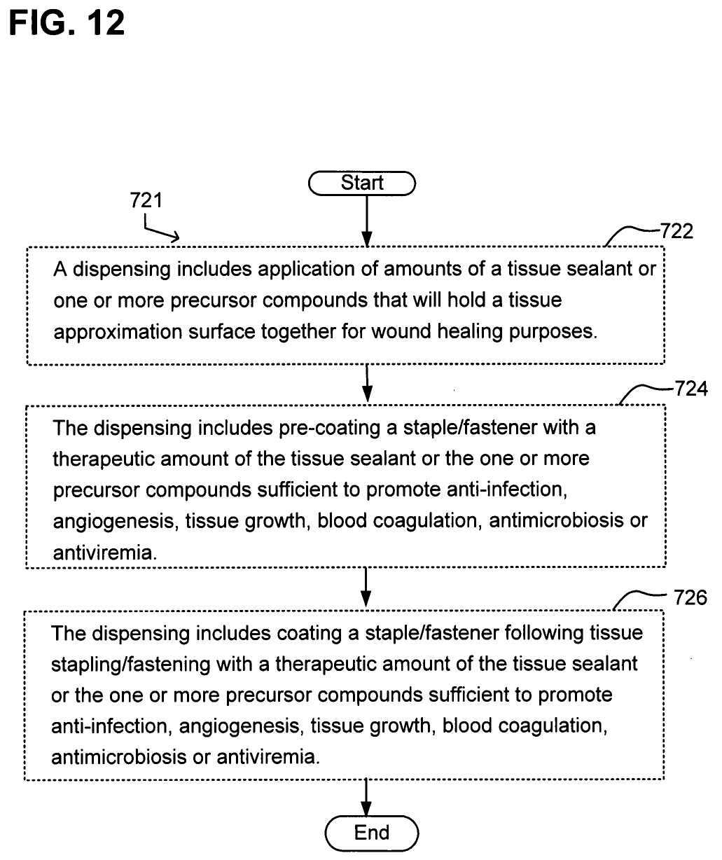

There is illustrated in FIG. 12 an example of an operational flow 721, wherein a dispensing step 722 includes an application of amounts of a tissue sealant or one or more precursor compounds that will hold a tissue approximation surface together for wound healing purposes. In another embodiment, the dispensing step 724 includes pre-coating a staple or fastener with a therapeutic amount of the tissue sealant or the one or more precursor compounds sufficient to promote anti-infection, angiogenesis, tissue growth, blood coagulation, antimicrobiosis or antiviremia. In yet another embodiment, the dispensing step 726 includes coating a staple or fastener following tissue stapling or fastening with a therapeutic amount of the tissue sealant or the one or more precursor compounds sufficient to promote anti-infection, angiogenesis, tissue growth, blood coagulation, antimicrobiosis or antiviremia.

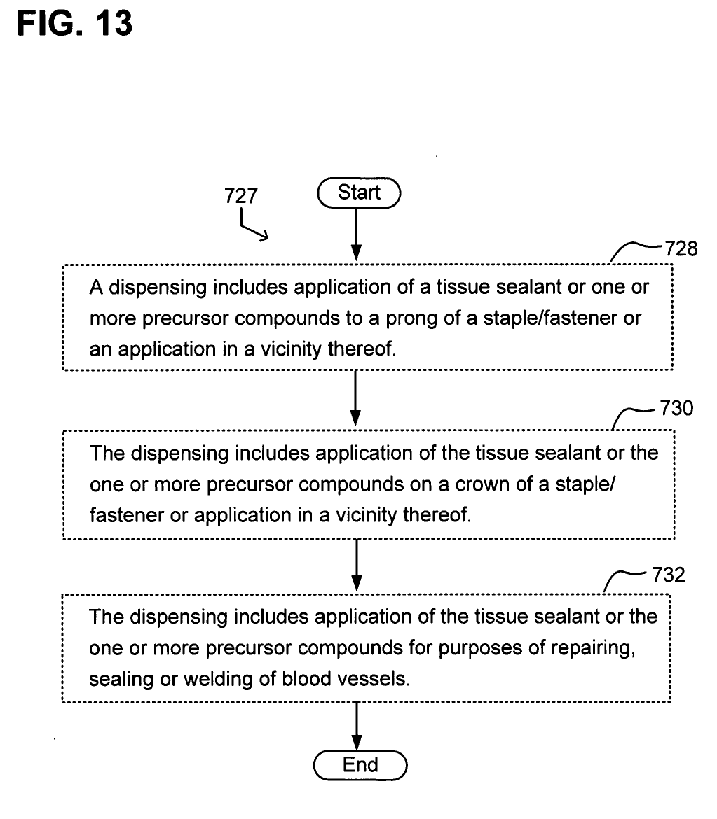

FIG. 13 shows an example of an operational flow 727 for additional optional steps in dispensing a tissue sealant. In one embodiment, the dispensing step 728 includes application of a tissue sealant or one or more precursor compounds to a prong of a staple or fastener or application in a vicinity thereof. Another embodiment provides that the dispensing step 730 includes application of the tissue sealant or the one or more precursor compounds on a crown of a staple or fastener or application in a vicinity thereof. Still another embodiment calls for the dispensing step 732 to include application of the tissue sealant or the one or more precursor compounds for purposes of repairing, sealing or welding of blood vessels. In an embodiment, a staple or a fastener may include one or more crowns.

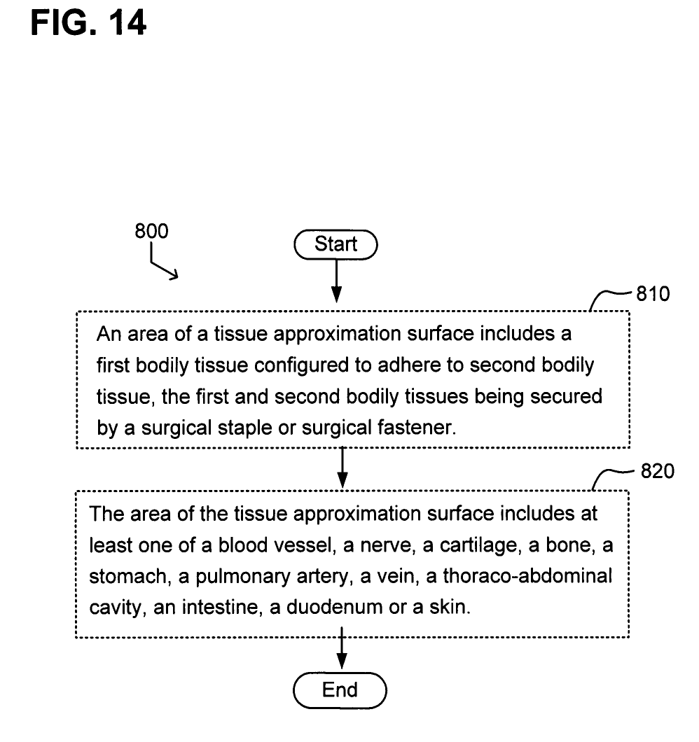

According to FIG. 14, an example of an operation 800 contains the following steps. In step 810, an area of a tissue approximation surface includes a first bodily tissue configured to adhere to second bodily tissue, the first and second bodily tissues being secured by a surgical staple or surgical fastener. Furthermore, the area of the tissue approximation surface includes at least one of a blood vessel, a nerve, a cartilage, a bone, a stomach, a pulmonary artery, a vein, a thoraco-abdominal cavity, an intestine, a duodenum or a skin.

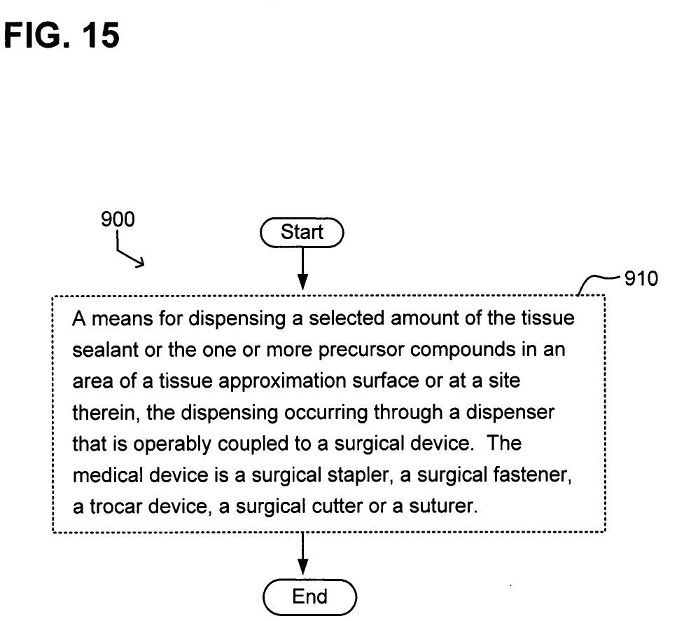

FIG. 15 illustrates an operational flow 900 for implementing embodiments of an example of a medical device. In an embodiment 910, the medical device comprises a means for dispensing a selected amount of the tissue sealant or the one or more precursor compounds in an area of a tissue approximation surface or at a site therein, the dispensing occurring through a dispenser that is operably coupled to a surgical device. In another embodiment 920, the medical device is a surgical stapler, a surgical fastener, a trocar device, a surgical cutter or a suturer.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

The foregoing detailed description has set forth various embodiments of the devices or processes via the use of flowcharts, diagrams, figures or examples. Insofar as such flowcharts, diagrams, figures or examples contain one or more functions or operations, it will be understood by those within the art that each function or operation within such flowchart, diagram, figure or example can be implemented, individually or collectively, by a wide range of any combination thereof.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

The herein described subject matter sometimes illustrates different components contained within, or connected with, different other components. It is to be understood that such depicted figures are merely by way of example, and that in fact many other figures can be implemented which achieve the same functionality. In a conceptual sense, any arrangement of components to achieve the same functionality is effectively “associated” or “coupled” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to, physically mateable or physically interacting components or wirelessly interactable or wirelessly interacting components or logically interacting or logically interactable components.

In a general sense, those skilled in the art will recognize that the various aspects described herein which can be implemented, individually or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes or devices described herein, or a microprocessor configured by a computer program which at least partially carries out processes or devices described herein), electrical circuitry forming a memory device (e.g., forms of random access memory) or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices or processes in the fashion set forth herein, and thereafter use engineering practices to integrate such described devices or processes into image processing systems. That is, at least a portion of the devices or processes described herein can be integrated into an image processing system via a reasonable amount of experimentation. Those having skill in the art will recognize that a typical image processing system generally includes one or more of a system unit housing, a video display device, a memory such as volatile and non-volatile memory, processors such as microprocessors and digital signal processors, computational entities such as operating systems, drivers, and applications programs, one or more interaction devices, such as a touch pad or screen, control systems including feedback loops and control motors (e.g., feedback for sensing lens position or velocity; control motors for moving or distorting lenses to give desired focuses). A typical image processing system may be implemented utilizing any suitable commercially available components, such as those typically found in digital still systems or digital motion systems.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that a limitation is desired.

With respect to the use of substantially any plural or singular terms herein, those having skill in the art can translate from the plural to the singular or from the singular to the plural as is appropriate to the context or application. The various singular or plural permutations are not expressly set forth herein for sake of clarity.

The herein described subject matter sometimes illustrates different components contained within, or connected with, different other components. It is to be understood that such depicted architectures are merely by way of example, and that in fact many other architectures can be implemented which achieve the same functionality. In a conceptual sense, any arrangement of components to achieve the same functionality is effectively “operably coupled” or “coupled” or “in communication with” or “communicates with” or “operatively communicate” such other objects that the desired functionality is achieved. Hence, any two components herein combined to achieve a particular functionality can be seen as associated with each other such that the desired functionality is achieved, irrespective of architectures or intermedial components. Likewise, any two components so associated can also be viewed as being “connected”, or “attached”, to each other to achieve the desired functionality, and any two components capable of being so associated can also be viewed as being “operably couplable”, to each other to achieve the desired functionality.

While particular aspects of the present subject matter described herein have been shown and described, it will be apparent to those skilled in the art that, based upon the embodiments herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). It will be further understood by those within the art that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.

1. A medical device, comprising:
a dispenser adapted to dispense a tissue sealant or one or more precursor compounds, said dispenser being operably coupled to a surgical stapler, wherein said surgical stapler is configured to deliver one or more surgical staples or fasteners in an area of bodily tissue approximation surface.

2. The medical device of claim 1, wherein said one or more surgical staples or fasteners have at least one coating or covering containing said tissue sealant or said one or more precursor compounds.

3. The medical device of claim 1, wherein said one or more surgical staples or fasteners are either pre-coated or post-coated with said tissue sealant or said one or more precursor compounds.

4. The medical device of claim 1, wherein said one or more surgical staples or fasteners are either partially coated or fully coated with said tissue sealant or said one or more precursor compounds.

5. The medical device of claim 1, wherein said tissue sealant is formed through at least one reaction that includes one or more of said one or more precursor compounds.

6. The medical device of claim 5, wherein said at least one reaction includes one of a reaction or a reaction with endogenous substrates, interaction with an endogenous clotting cascade of a bloodstream, a photo reaction with either a endogenously-generated photons or photons generated external to a bodily tissue or a thermally-driven reaction or a catalytically-activated reaction.

7. (canceled)

8. (canceled)

9. The medical device of claim 1, wherein said dispenser is operably configured to dispense microfluidic amounts of said tissue sealant or said one or more precursor compounds.

10. The medical device of claim 1, wherein said dispenser includes one or more micropipettes that dispense said tissue sealant or said one or more precursor compounds.

11. The medical device of claim 1, wherein said dispenser includes one or more micron-sized outlet ports that dispense said tissue sealant or one or more precursor compounds.

12. The medical device of claim 1, wherein said dispenser includes one or more microchips containing said tissue sealant or said one or more precursor compounds.

13. The medical device of claim 1, wherein said dispenser includes one or more arrays of microchips containing more than one type of tissue sealant or said one or more precursor compounds.

14. The medical device of claim 1, wherein said dispenser includes one or more arrays of micropipettes that dispense more than one type of tissue sealant or said one or more precursor compounds.

15. (canceled)

16. (canceled)

17. The medical device of claim 1, wherein said tissue sealant or said one or more precursor compounds include at least one of antibacterial agents, anti-infection agents, angiogenic factors, growth factors, blood coagulants, antimicrobial agents, pharmaceuticals, drugs or compounds.

18. The medical device of claim 1, wherein said tissue sealant or said one or more precursor compounds reduces scar formation in body tissue.

19. The medical device of claim 1, wherein said tissue sealant or said one or more precursor compounds include at least one of an acrylic acid-derivative.

20. The medical device of claim 1, wherein said tissue sealant or said one or more precursor compounds include at least one of a gel, a cream, a liquid, a fluid, a semi-solid or solid.

21. The medical device of claim 1, wherein said tissue sealant or said one or more precursor compounds include at least one of a hydrogel, an alginate, a zymogen, a glutaraldehyde-treated protein, a cross-linked protein, a cross-linked carbohydrate or a cross-linked fatty acid derivative.

22-125. (canceled)

126. A method of applying tissue sealant to an area of a tissue approximation surface comprising:
dispensing a selected amount of said tissue sealant or one or more precursor compounds, said dispensing occurring through a dispenser that is operably coupled to a surgical device.

127. The method of claim 126, wherein said surgical device is a surgical stapler, a surgical fastener, a trocar device, a surgical cutter or a suturer.

128. (canceled)

129. The method of claim 126, wherein said dispensing includes location of a site of at least one surgical cut, a surgical suture or a trocar site, or a needle puncture site, or a drain or an entry site of a percutanously inserted or deployed medical device sites are covered with said tissue sealant or said one or more precursor compounds in a body tissue.

130. (canceled)

131. The method of claim 126, wherein said dispensing includes application of therapeutic amounts of said tissue sealant or said one or more precursor compounds for purposes of inducing anti-infection response, angiogenesis, promoting tissue growth, enhancing blood coagulation, antimicrobial activity or antiviral response.

132. The method of claim 126, wherein said dispensing includes application of amounts of said tissue sealant or said one or more precursor compounds that will hold said tissue approximation surface together for wound healing purposes.

133. The method of claim 126, wherein said dispensing includes pre-coating a staple or fastener with a therapeutic amount of said tissue sealant or said one or more precursor compounds sufficient to promote anti-infection, angiogenesis, tissue growth, blood coagulation, antimicrobiosis or antiviremia.

134. The method of claim 126, wherein said dispensing includes coating a staple or fastener following tissue stapling or fastening with a therapeutic amount of said tissue sealant or said one or more precursor compounds sufficient to promote tissue repair, welding blood vessels, anti-infection, angiogenesis, tissue growth, blood coagulation, antimicrobiosis or antiviremia.

135-137. (canceled)

138. The method of claim 126, wherein said area of a tissue approximation surface includes at least one of a blood vessel, a nerve, a cartilage, a bone, a stomach, a pulmonary artery, a vein, a thoraco-abdominal cavity, an intestine, a duodenum, a skin, a fascia, a dermis, a muscle, a meningeal layer, a bowel wall or a mucosal layer.

139-153. (canceled)
